(19) United States
(12) Patent Application Publication
(10) Pub. No.: US 2025/0345202 A1
(43) Pub. Date: Nov. 13, 2025

(54) ORAL CONDOM

(71) Applicant: CIAN-SIN WU, Kaohsiung City (TW)

(72) Inventor: CIAN-SIN WU, Kaohsiung City (TW)

(21) Appl. No.: 19/192,738

(22) Filed: Apr. 29, 2025

(30) Foreign Application Priority Data
May 13, 2024 (TW) 113204864

(51) Int. Cl. A61F 6/04

(52) U.S. Cl. CPC A61F 6/04; A61F 2006/042; A61F 2006/045; A61F 2006/047; A61F 2006/048

(57) ABSTRACT

An oral condom is configured to be worn on a user's mouth, lips and tongue, and the oral condom has a membrane and a stereoscopic pocketed structure. The membrane is an elongated film and has high-density elasticity, and the membrane may be wrapped around a backside of the user's head. The membrane has two through-holes respectively formed at two ends of the membrane. The stereoscopic pocketed structure protrudes from the membrane, and the stereoscopic pocketed structure is located in a center of the membrane and is capable of covering the user's mouth, lips and tongue. The user's tongue can stretch up, down, left, and right in the stereoscopic pocketed structure blocking the body fluids and secretions of the sexual partner during sexual intercourse. The stereoscopic pocketed structure may comprise a smooth surface, or the stereoscopic pocketed structure comprises an uneven texture or a grained texture.

### FIELD

The present invention relates to an oral condom configured to be worn on a user's mouth, lips and tongue.

### BACKGROUND

The conventional condom is only used on the sexual organ or only uses a flat sheet to cover the sexual organ. However, viruses transmitted through the mouth and the tongue, such as human papillomavirus, immunocompromised viruses, and common sexual diseases, cause more and more cancers in the mouth, tongue, esophagus and cervix, but there is a lack of formal protective measures.

To overcome the shortcomings, the present invention provides an oral condom to mitigate or obviate the aforementioned problems.

### SUMMARY

Though with human papillary vaccination and the popularization of modern medicine, most people still neglect necessary precautions, and thus the number of people having cancers remains unabated. Therefore, an oral condom in accordance with the present invention provides more auxiliary and preventive measures in daily life, so as to reduce disease infections and increase the safety and pleasure during sexual intercourse.

The oral condom is configured to be worn on a user's mouth, lips and tongue, and the oral condom comprises a membrane and a stereoscopic pocketed structure. The membrane is an elongated film having high-density elasticity, and the membrane may be wrapped around a backside of the user's head. The membrane comprises two through-holes respectively formed at two ends of the membrane.

The stereoscopic pocketed structure protrudes from the membrane, and the stereoscopic pocketed structure is located in a center of the membrane and is capable of covering the user's mouth, lips and tongue.

The main objective of the present invention is to prevent sexually-transmitted diseases and provide protection. Basically, as most of the conventional preventive measures are just a sheet, the present invention not only blocks transmission of sexual diseases but also adds safety and security during sexual intercourse, saving more medical costs. Therefore, the oral condom provides basic protective measures during sexual intercourse and reduces the burden of the medical system.

Other objectives, advantages and novel features of the invention will become more apparent from the following detailed description when taken in conjunction with the accompanying drawings.

### BRIEF DESCRIPTION OF THE DRAWINGS

FIG. 1 is a schematic bottom view of an oral condom in accordance with the present invention;

FIG. 2 is a front view of the oral condom in FIG. 1;

FIG. 3 is a rear view of the oral condom in FIG. 1;

FIG. 4 is a side view of the oral condom in FIG. 1;

FIG. 5 is a side view of the oral condom in FIG. 1;

FIG. 6 is a perspective view of two through-holes embedded in each other of the oral condom in FIG. 1; and

FIG. 7 is a partial cross-sectional view of a membrane of the oral condom in FIG. 1.

### DETAILED DESCRIPTION OF THE PREFERRED EMBODIMENTS

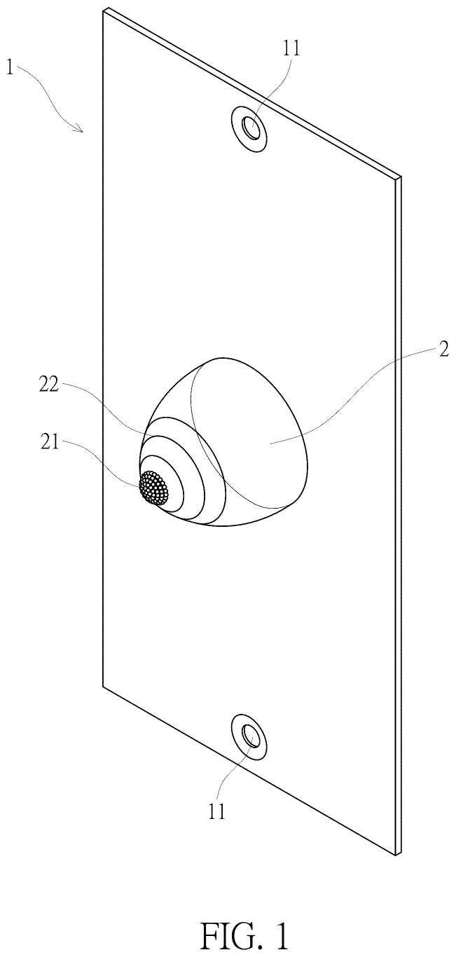

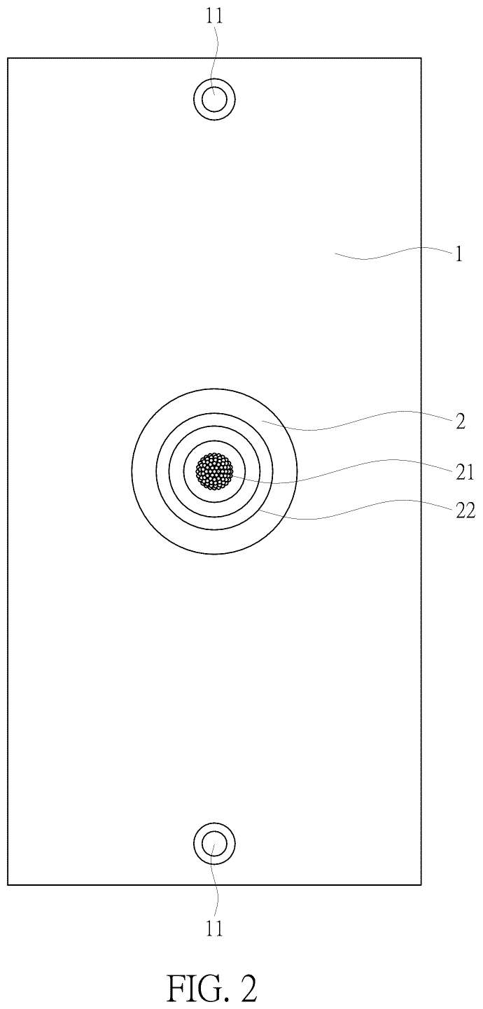

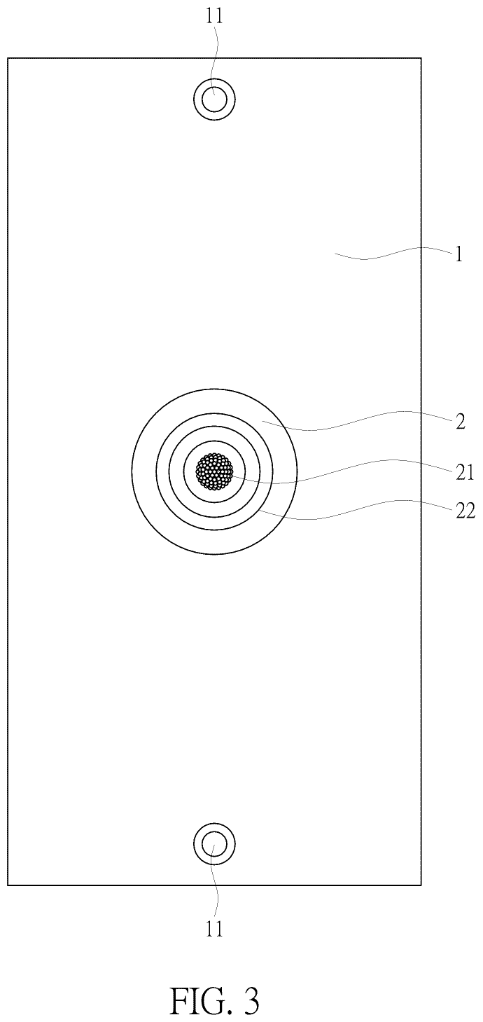

With reference to FIG. 1 to FIG. 3, an oral condom in accordance with the present invention is configured to be worn on a user's mouth, lips and tongue. The oral condom comprises a membrane 1 and a stereoscopic pocketed structure 2, and selectively comprises a hook 3. The membrane 1 is an elongated film and has high-density elasticity. The membrane 1 is wearable on a user's mouth, lips and tongue. An upper edge of the membrane is configured to cover the user's nose, and a lower edge of the membrane is configured to cover the user's chin to secure the position of the membrane, so that the oral condom may not roll upward. The membrane 1 may made by latex, synthetic rubber, or polyurethane, but it is not limited thereto.

With reference to FIG. 1 to FIG. 3 and FIG. 6, the membrane 1 comprises two through-holes 11 respectively formed at two ends of the membrane 1. The hook 3 is detachably mounted through the two through-holes 11, such that the membrane 1 can wrap around a backside of the user's head and the two ends of the membrane 1 can lock each other with the hook 3. When the two ends of the membrane 1 lock each other with the hook 3, the hook 3 can support and fix the membrane 1, and the user can put on or take off the oral condom by the through-holes 11 and the hook 3. In another embodiment, the oral condom may not comprise the hook 3, and the membrane 1 can be fixed by other accessories or means. In this embodiment, the hook 3 is S-shaped, and the two ends of the hook 3 can be fastened to the two through-holes 11 of the membrane 1. In another embodiment, the configuration of the hook 3 is not limited thereto, and the hook 3 may be a ring, as long as the hook 3 can fix the two ends of the membrane 1.

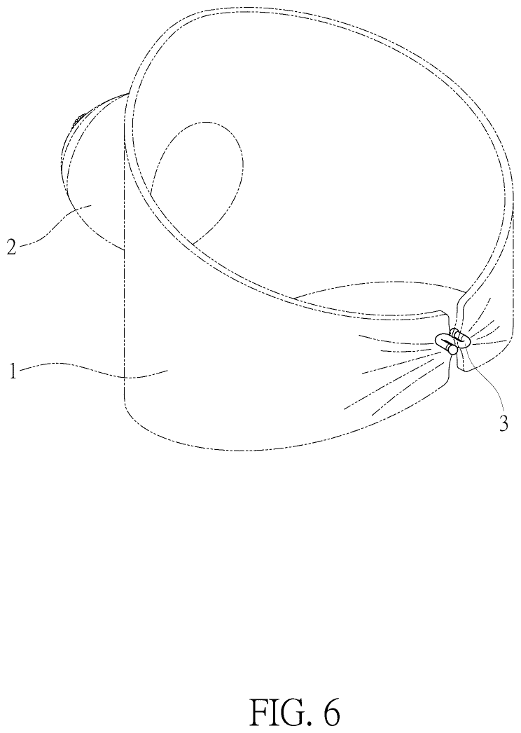

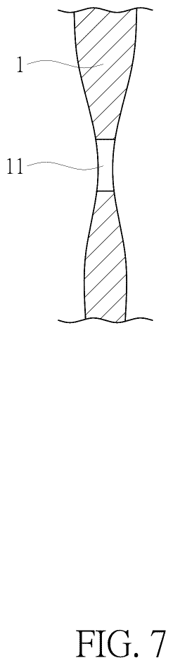

With reference to FIG. 1 to FIG. 3, FIG. 6, and FIG. 7, in this embodiment, because the membrane 1 has elasticity, when the user stretches the membrane 1 to get the two through-holes 11 approaching each other, the width of the membrane 1 around the through-holes 11 will be reduced due to the stretching and wrinkles are produced, so that the strength around the through-holes 11 will be increased. In addition, a thickness of the membrane 1 around the two through-holes 11 is thicker than a thickness of any other portion of the membrane 1, which can also increase the tolerance of the membrane 1 around the two through-holes 11 and the membrane 1 around the two through-holes 11 is not prone to breakage during sexual intercourse.

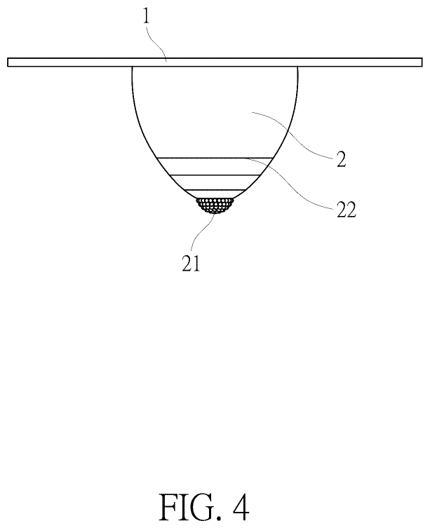

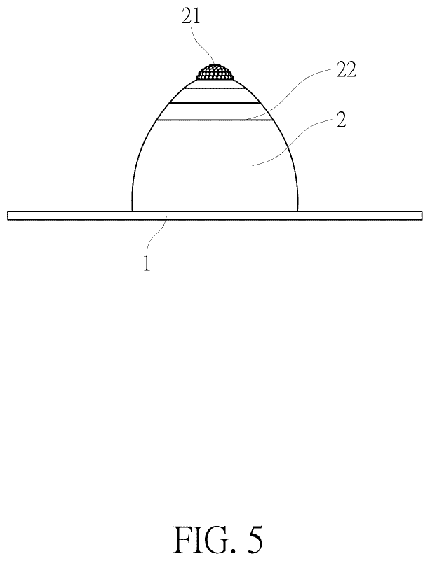

With reference to FIG. 1, FIG. 4 and FIG. 5, the stereoscopic pocketed structure 2 is mounted on the front side of the membrane 1 and corresponds to the user's mouth, lips and tongue in position and size. The user's tongue can stretch up, down, left, and right in the stereoscopic pocketed structure 2 blocking the body fluids and secretions of the sexual partner during sexual intercourse. Precisely, the membrane 1 and the stereoscopic pocketed structure 2 can together form a seamless vertical isolation protective bladder. In another embodiment, a depth and a width of the stereoscopic pocketed structure 2 are not limited thereto and can be adjusted according to the sexual partner and the user.

In this embodiment, the stereoscopic pocketed structure 2 may comprise a smooth surface, or may comprise an uneven texture 21 or a grained texture 22, adding the touch of the oral condom and increasing the erotic pleasure.

In another embodiment, the oral condom may be colored such as fluorescent color. The oral condom may be flavored, and the flavor can be fruit flavors, such as peach, apple, or human hormone, selectable by the user.

The advantage of the present invention is that the membrane 1 and the stereoscopic pocketed structure 2 can block the direct contact with the body fluids and secretions of the sexual partner during sexual intercourse, making the user safer and saving more medical costs. Therefore, the oral condom not only provides basic protective measures during sexual intercourse but also reduces the burden of the medical system.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An oral condom configured to be worn on a user's mouth, lips and tongue, and the oral condom comprising:
a membrane being an elongated film and having high-density elasticity, and the membrane capable of being wrapped around a backside of the user's head; the membrane comprising:
two through-holes respectively formed at two ends of the membrane;
a stereoscopic pocketed structure protruding from the membrane, and the stereoscopic pocketed structure located in a center of the membrane and being capable of covering the user's mouth, lips and tongue.

2. The oral condom as claimed in claim 1 further comprising:
a hook detachably mounted through the two through-holes, such that the membrane is capable of being wrapped around the backside of the user's head.

3. The oral condom as claimed in claim 1, wherein, an upper edge of the membrane is configured to cover the user's nose and a lower edge of the membrane is configured to cover the user's chin to secure the oral condom without rolling upward.

4. The oral condom as claimed in claim 2, wherein, an upper edge of the membrane is configured to cover the user's nose and a lower edge of the membrane is configured to cover the user's chin to secure the oral condom without rolling upward.

5. The oral condom as claimed in claim 1, wherein, the stereoscopic pocketed structure comprises a smooth surface, or the stereoscopic pocketed structure comprises an uneven texture or a grained texture.

6. The oral condom as claimed in claim 4, wherein, the stereoscopic pocketed structure comprises a smooth surface, or the stereoscopic pocketed structure comprises an uneven texture or a grained texture.

7. The oral condom as claimed in claim 1 being colored or flavored.

8. The oral condom as claimed in claim 6 being colored or favored.

9. The oral condom as claimed in claim 1, wherein, a thickness of the membrane around the two through-holes is thicker than a thickness of any other portion of the membrane.

10. The oral condom as claimed in claim 8, wherein, a thickness of the membrane around the two through-holes is thicker than a thickness of any other portion of the membrane.
